(19) United States
(12) Patent Application Publication
(10) Pub. No.: US 2012/0143191 A1
(43) Pub. Date: Jun. 7, 2012

(54) JOINT FIXATOR

(76) Inventor: Brian Foote, Blackburn (GB)

(21) Appl. No.: 12/519,825

(22) PCT Filed: Dec. 17, 2007
(86) PCT No.: PCT/GB2007/050761

§ 371 Date: Dec. 29, 2011

(30) Foreign Application Priority Data
Dec. 20, 2006 (GB) 0625681.2

(51) Int. Cl. A61B 17/64

(52) U.S. Cl. 606/59

(57) ABSTRACT

Disclosed is a fixator (1) for treating a fracture dislocation injury, comprising: first (10) and second elements for piercing first and second (20) bones, separated by a joint; and first and second separators (30) arranged to engage with free ends of the first and second elements and to be variable in length.

The present invention is concerned with a joint fixator, for use particularly, but not exclusively, in relation to reduction of fracture dislocations affecting proximal interphalangeal joint (the middle joint of the finger).

When a finger is injured, it can be broken in one of a number of different ways. One particular injury which can occur is a fracture dislocation. This is where a bone is broken, but the joint close to that bone is also dislocated. Such an injury presents conflicting clinical needs: it is desirable to immobilise the bone to aid healing, whilst it is similarly desirable to allow the patient to mobilise the joint to prevent stiffness developing.

Such a conflict means that simply plastering or splinting the injured finger is not particularly effective, as the joint can then become stiff and sore, and further complications such as long-term pain can arise.

Prior art attempts to solve this problem have involved the use of various forms of splint, some of which are positioned percutaneously and some not. However, none of these has proved particularly successful.

Some prior art devices require the use of external biasing devices, such as elastic bands to maintain a particular configuration of fixator. These can be troublesome in use and the amount of flexure available can be limited.

Embodiments of the present invention aim to address problems with the prior art, whether mentioned herein or not.

According to the present invention there is provided an apparatus as set forth in the appended claims. Preferred features of the invention will be apparent from the dependent claims, and the description which follows.

For a better understanding of the invention, and to show how embodiments of the same may be carried into effect, reference will now be made, by way of example, to the accompanying diagrammatic drawings in which:

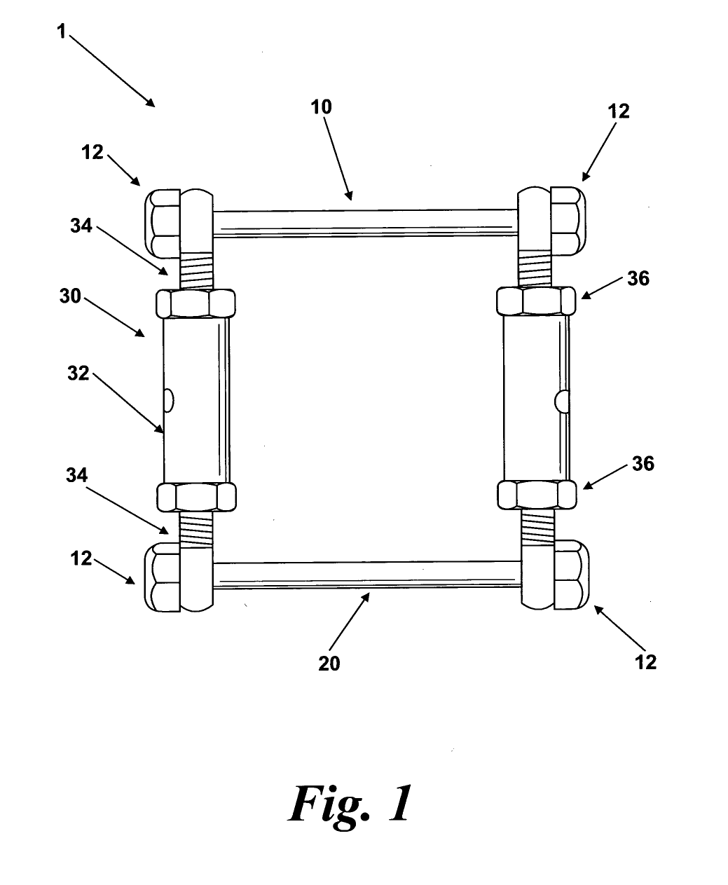

FIG. 1 shows a view of an embodiment of the present invention;

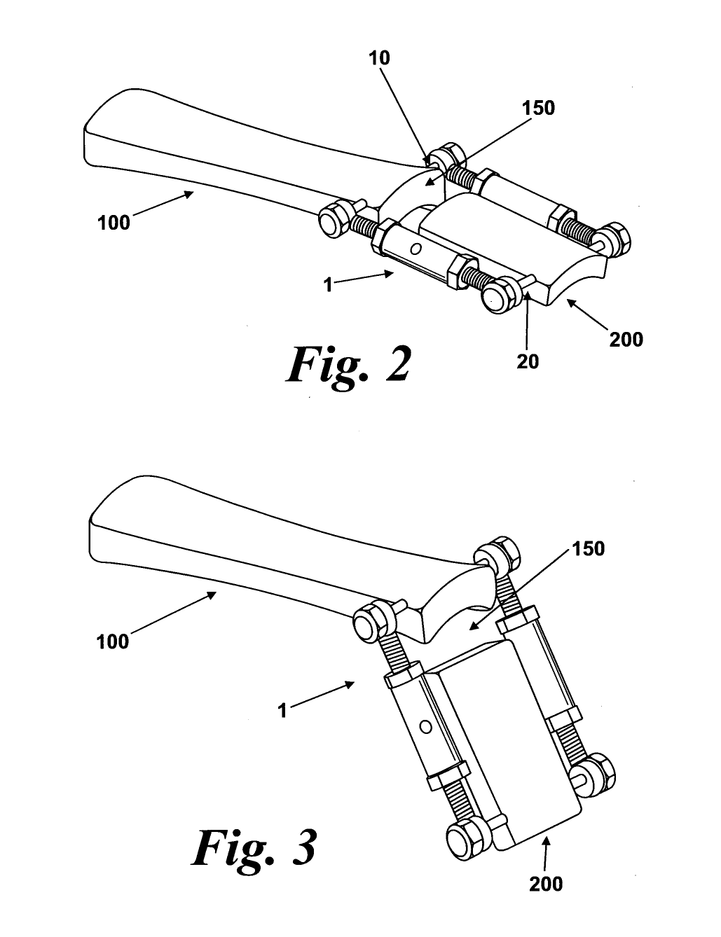

FIG. 2 shows a representation of an embodiment of the invention in-situ, with the finger straight; and

FIG. 3 shows a representation of an embodiment of the invention in-situ, with the finger bent.

Embodiments of the present invention offer several advantages over prior art solutions. They allow a fractured bone to be immobilised whilst still allowing an associated joint to me mobilised, thus aiding the rehabilitation of the joint.

As can be seen in FIGS. 1 to 3, a fixator 1, according to an embodiment of the invention, comprises a first element 10 for passing through the proximal phalanx 100 and a second element 20 for passing through the middle phalanx 200. The first and second elements are each composed of a relatively short length of metallic material, known as Kirschner wire (or K wire). K wire elements are composed of stainless steel and are sharpened to ease penetration into the subject bone.

The first and second elements 10, 20 are positioned percutaneously and through the respective bones 100, 200 referred to above. The first element 10 is located so that it passes through the end of the proximal phalanx 100 closest to the middle phalanx 200 and at a point as close as possible to the natural axis of rotation between the respective bones.

The second element 20 is located so that it passes through the middle phalanx 200 at a suitable point, some distance from the proximal inter-phalangeal joint 150.

Both first and second elements are positioned under local, or possibly general, anaesthetic. Alternatively, a Bier block, which induces a powerful localised anaesthesia may be used. The sharpened K-wire is able to pierce the skin and flesh easily, but drilling may be required to penetrate the bone.

Once the first and second K-wire elements 10, 20 are inserted, a separator 30 is positioned over each of their free ends, which protrude from the finger. The separator 30 is of the form of a bottle screw or turnbuckle, and comprises an elongate barrel body 32 having a threaded aperture at each end. Inserted into each of the threaded apertures is a correspondingly threaded eyelet 34. The eyelet portion engages with each free end of the first and second elements, as shown in FIGS. 2 and 3.

The apertures at each end of the barrel body 32 are configured to be oppositely threaded (i.e. one is a left-handed thread and the other is a right-handed thread) so that by rotating the barrel body 32 relative to the threaded eyelets 34, each of the threaded eyelets 34 are simultaneously drawn into or urged out of the barrel body, so that the overall length of the separator can be increased or decreased as desired.

The barrel body 32 and threaded eyelets 34 comprise stainless steel.

Once the aforementioned parts have been assembled, an end cap 12 is added to the exposed ends of each of the first and second elements. The end cap is provided to secure the separator 30 in position and to ensure that the possibly sharp ends of the first and second elements 10, 20 are covered and unable to damage the adjacent fingers.

The end caps 12 are cups filled with a resiliently deformable material, such that when the end of the first or second element 10, 20 is pushed into the material, the caps are retained and held in position. The cups may comprise a metal, such as steel or aluminium, or may comprise a plastics material. The resiliently deformable material may be a natural or synthetic rubber material.

Once the device 1 has been fitted to the patient, the clinician can fine-tune the fixator 1 to achieve the required separation between the proximal phalanx 100 and middle phalanx 200. The separation is adjusted by rotating each barrel 32, causing the threaded eyelets 34 to move relatively in or out as required. Once the desired separation is achieved, Each of the two pairs of lock nuts 36 are tightened to prevent inadvertent alteration of the fixator 1.

With the fixator 1 in position, the injured finger may be flexed while the fracture is healing, unlike prior art solutions where this is generally not possible.

Since the axis of rotation is defined by the placement of the first element 10, it should be placed so that the separation between phalanges 100, 200 is substantially constant over the full flexure of the inter-phalangeal joint. In effect, the first element should be positioned as close to the end of the proximal phalanx as possible.

Over time, as the fracture heals, a clinical assessment can be made of the need to adjust the fixator to vary the separation between the phalanges 100, 200. Over time, the distance can be reduced so that the separation tends towards the norm.

After a period of time, which may be days or weeks, depending on the patient's progress, the fixator is adjusted so that the separation returns to normal as the fracture heals. Once a decision has been made to remove the device 1, the external parts are removed and the first and second elements 10, 20 are withdrawn under local anaesthetic.

Although the present invention has been described with particular reference to an injury affecting the proximal interphalangeal joint, it will be understood that other phalangeal injuries could be treated using an embodiment of the invention. Furthermore, larger-scale embodiments of the invention could also be used to fixate and reduce other hinged joints such as knees or elbows.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A fixator for treating a fracture dislocation injury, comprising:
first and second elements for piercing first and second bones, separated by a joint; and
first and second separators arranged to engage with free ends of the first and second elements and to be variable in length.

2. The fixator of claim 1 wherein the first and second separators each comprise an elongate barrel body having an oppositely threaded aperture at each end, and a threaded eyelet for co-operation with each threaded aperture.

3. The fixator of claim 2 comprising a locking device to lock the length of the separator.

4. The fixator of claim 3 wherein the locking device comprises a pair of locking nuts.

5. The fixator of claim 1 further comprising an end cap, associated with each free end of the first and second elements.

6. The fixator of claim 1 wherein the first and second elements comprise Kirschner wire.

7. The fixator of claim 1 wherein the separators comprise stainless steel.

8. (canceled)
